(19) United States
(12) Patent Application Publication
(10) Pub. No.: US 2006/0233883 A1
(43) Pub. Date: Oct. 19, 2006

(54) Intravenous nanoparticles for targeting drug delivery and sustained drug release

(76) Inventors: Tsutomu Ishihara, Tokyo (JP); Yutaka Mizushima, Tokyo (JP)

Correspondence Address: OSTRAGER CHONG FLAHERTY & BROITMAN PC, 250 PARK AVENUE, SUITE 825, NEW YORK, NY, 10177, US

(21) Appl. No.: 10/550,990

(22) PCT Filed: Mar. 11, 2004
(86) PCT No.: PCT/JP04/03246

§ 371 Date: Sep. 26, 2005

(30) Foreign Application Priority Data
Mar. 26, 2003 (JP) 2003-084695

(51) Int. Cl. A61K 9/50; A61K 33/24; A61K 33/34; A61K 9/16; A61K 33/26; A61K 31/573

(52) U.S. Cl. 424/490; 424/638; 424/641; 424/617; 424/646; 977/906; 514/171

(57) ABSTRACT

Provided are poly(lactic-co-glycolic acid) (PLGA) and poly(lactic acid) (PLA) nanoparticles that encapsulate a low molecular weight and water-soluble drug and can deliver the drug to target legion sites where the particles gradually release the drug over a prolonged period of time. The nanoparticles are prepared by allowing the low-molecular, water-soluble and non-peptide drug to interact with a metal ion so as to make the drug hydrophobic, encapsulating the hydrophobicized drug into PLGA or PLA nanoparticles, and allowing a surfactant to be adsorbed onto the surface of the particles.

### TECHNICAL FIELD

The present invention relates to intravenous nanoparticles encapsulating low-molecular weight, water-soluble and non-peptide drugs that are intended for the purposes of targeting drug delivery and sustained drug release. The invention also relates to a production method of such nanoparticles. Specifically, the present invention relates to intravenous nanoparticles which can deliver low-molecular weight, water-soluble and non-peptide drugs to target lesion site where the particles gradually release the drugs over a prolonged period of time, and a production method thereof. Hereon, intravenous nanoparticles mean nanoparticles for intravenous administration containing drugs.

### BACKGROUND ART

Many researchers have developed and proposed poly(lactic-co-glycolic acid) (PLGA) or poly(lactic acid) (PLA) microparticles and nanoparticles that encapsulate low-molecular weight, water-soluble drugs.

For example, U.S. Pat. No. 4,652,441 describes PLGA microcapsules containing physiologically active polypeptides and a production method thereof. Japanese National Publication No. Hei 10-511957 describes PLGA nanoparticles for intravascular administration containing various therapeutic agents. Also, Japanese Patent Laid-Open Publication No. Hei 8-217691 discloses a sustained-release formulation comprising PLGA microcapsules encapsulating physiologically active, water-soluble peptide compounds, which were prepared in the form of water-insoluble or hardly water-soluble polyvalent metal salts.

However, none of these patent publications mention or suggest the concept of hydrophobicizing a low-molecular weight, water-soluble and non-peptide drug with the use of metal ions prior to the encapsulation of the drug into nanoparticles so as to make intravenous nanoparticles suitable for the targeting delivery and sustained release of drugs.

The present inventors also have filed patent applications (e.g., Japanese Patent Application No. 2002-159190) concerning formulations comprising poly(lactic-co-glycolic acid) (PLGA) or poly(lactic acid) (PLA) nanoparticles. The nanoparticles suggested by the present inventors, however, could only offer a low encapsulation efficiency of the low-molecular weight, water-soluble drugs. Attempts were therefore made to increase the hydrophobicity and thereby the encapsulation rate of the drug through processes including esterification. However, such attempts resulted in a decrease in the length of time over which the nanoparticles can release the encapsulated drug, though the encapsulation rate was improved to some extent. In other words, the desired sustained drug-releasing property of the nanoparticles was compromised in these approaches.

Accordingly, it is an objective of the present invention to provide intravenous nanoparticles encapsulating a low-molecular weight, water-soluble and non-peptide drug that are capable of targeting a specific lesion site and are less likely to burst at an early stage of administration so that they can gradually release the drug at the lesion site over a prolonged period of time.

It is another objective of the present invention to provide a simple method for preparing such intravenous nanoparticles that enables large-scale production of the product.

In an effort to attain the above-described goals, the present inventors drew attention to the fact that low-molecular weight, water-soluble and non-peptide drugs interact with certain metal ions. Specifically, the present inventors have examined the possibility of allowing such low-molecular weight, water-soluble and non-peptide drugs to bind to metal ion to impart a hydrophobicity to the drugs, thereby facilitating encapsulation of the drugs into PLGA or PLA nanoparticles. As a result, the present inventors have discovered that such drugs, when bound to a metal ion, become hydrophobic and thus can be readily encapsulated in PLGA or PLA nanoparticles. In fact, it has proven that the encapsulation efficiency of the incorporation of the hydrophobicized drugs into the nanoparticles was extremely high. The present inventors have also discovered that the nanoparticles so produced have an ability to gradually release the drugs over time and tend to accumulate specific lesion sites in living body. This implied the possibility that such nanoparticles can be suitably used in designing a formulation for effective targeted drug delivery and sustained drug release. The discoveries ultimately led the present inventors to devise the present invention.

### DISCLOSURE OF THE INVENTION

Accordingly, one aspect of the present invention concerns intravenous nanoparticles designed for targeting drug delivery and sustained drug release. The nanoparticles are characterized in that a low-molecular weight, water-soluble and non-peptide drug is made hydrophobic by a metal ion and is encapsulated in nanoparticles formed of poly(lactic-co-glycolic acid) (PLGA) or poly(lactic acid) (PLA), and a surfactant is applied to the surface of the PLGA or PLA nanoparticles.

In one specific embodiment of the intravenous nanoparticles according to the present invention, the PLGA or PLA nanoparticles has a diameter of 50 to 300 nm.

In one specific embodiment of the intravenous nanoparticles according to the present invention, the low-molecular weight, water-soluble and non-peptide drug to be encapsulated in the PLGA or PLA nanoparticles has a molecular weight of 1000 or lower.

In another specific embodiment of the intravenous nanoparticles according to the present invention, the metal ion to be bound to the low-molecular weight, water-soluble and non-peptide drug is any of zinc, iron, copper, nickel, beryllium, manganese, and cobalt.

In a further specific embodiment of the intravenous nanoparticles according to the present invention, the low-molecular weight, water-soluble and non-peptide drug to be encapsulated in the PLGA or PLA nanoparticles has a phosphate group or a carboxyl group in its molecule.

In a still more specific embodiment of the intravenous nanoparticles according to the present invention, the low-molecular weight, water-soluble and non-peptide drug is a steroidal anti-inflammatory agent, a non-steroidal anti-inflammatory agent, a prostanoid, an antimicrobial agent, or an anticancer agent.

In a still more specific embodiment of the intravenous nanoparticles, the surfactant to coat the surface of the PLGA or PLA nanoparticles encapsulating the low-molecular weight, water-soluble and non-peptide drug is a polyoxyethylene polyoxypropylene glycol, a polysorbate, a polyoxyethylene octylphenyl ether, a lecithin, or a polyvinylalcohol.

Another aspect of the present invention concerns a method for producing intravenous nanoparticles for targeting drug delivery and sustained drug release. Specifically, the method comprises the steps of hydrophobicizing a low-molecular weight, water-soluble and non-peptide drug by the use of metal ion; dissolving or suspending, along with PLGA or PLA, the low-molecular weight, non-peptide drug in a water-miscible organic solvent; and adding the resulting solution or the suspension to an aqueous solution of a surfactant to apply the surfactant to the surface of the PLGA or PLA nanoparticies.

In one specific embodiment of the method for producing intravenous nanoparticles according to the present invention, the resulting PLGA or PLA particles have a diameter 50 to 300 nm.

In another specific embodiment of the method for producing intravenous nanoparticles according to the present invention, the low-molecular weight, water-soluble and non-peptide drug to be encapsulated in the PLGA or PLA nanoparticles has a molecular weight of 1000 or lower.

In a further specific embodiment of the method for producing intravenous nanoparticles according to the present invention, the metal ion to be bound to the low-molecular weight, water-soluble and non-peptide drug is any of zinc, iron, copper, nickel, beryllium, manganese, and cobalt.

In a still more specific embodiment of the method for producing intravenous nanoparticles according to the present invention, the low-molecular weight, water-soluble and non-peptide drug to be encapsulated in the PLGA or PLA nanoparticles has a phosphate group or a carboxyl group in its molecule.

In a still more specific embodiment of the method for producing intravenous nanoparticles according to the present invention, the low-molecular weight, water-soluble and non-peptide drug is a steroidal anti-inflammatory agent, a non-steroidal anti-inflammatory agent, a prostanoid, an antimicrobial agent, or an anticancer agent.

In a still more specific embodiment of the method for producing intravenous nanoparticles according to the present invention, the surfactant to coat the surface of the PLGA or PLA nanoparticles encapsulating the low-molecular weight, water-soluble and non-peptide drug is a polyoxyethylene polyoxypropylene glycol, a polysorbate, a polyoxyethylene octylphenyl ether, a lecithin, or a polyvinylalcohol.

Another aspect of the present invention concerns a therapeutic preparation containing as an active ingredient the above-described nanoparticles. Specifically, the therapeutic preparation is an anti-inflammatory/anti-rheumatoid agent containing as an active ingredient the nanoparticles encapsulating a water-soluble steroid.

### BEST MODE FOR CARRYING OUT THE INVENTION

As described above, the present invention comprises biodegradable PLGA or PLA nanoparticles; a low-molecular weight, water-soluble and non-peptide drug bound to a metal ion and encapsulated in the nanoparticles; and a surfactant applied to the surfaces of the nanoparticles.

Specifically, the intravenous nanoparticles of the present invention designed for targeting drug delivery and sustained drug release comprise a low-molecular weight, water-soluble and non-peptide drug that has been hydrophobicized with a metal ion and has been encapsulated in PLGA or PLA nanoparticles with a surfactant subsequently applied to their surfaces.

In this regard, it has been found that the nanoparticles of the present invention are most effectively uptaken by the target lesion site when they have a diameter of 50 to 300 nm. The nanoparticles having a diameter less than 50 nm tend to be uptaken by regions other than the intended lesion sites and are therefore undesirable, as are the nanoparticles having a diameter larger than 300 nm, which tend to be uptaken by endothelial cells.

One characteristic feature of the present invention is that the low-molecular weight, water-soluble and non-peptide drug is bound to a metal ion so that the low-molecular weight drug will become hydrophobic and is thus effectively encapsulated in the nanoparticles. Among the metal ions suitable for this purpose are zinc ion, iron ion, copper ion, nickel ion, beryllium ion, manganese ion, and cobalt ion. Of these, zinc ion and iron ion are particularly preferred.

For this reason, the low-molecular weight, water-soluble and non-peptide drug to be encapsulated in the PLGA or PLA nanoparticles in accordance with the present invention preferably includes a phosphate group or a carboxyl group in its molecule so that the drug can readily bind to the metal ion to become hydrophobic.

Preferably, the low-molecular weight, water-soluble and non-peptide drug has a molecular weight of 1000 or less.

While various drugs can be used as the low-molecular weight, water-soluble and non-peptide drug in the present invention, particularly preferred are water-soluble steroidal anti-inflammatory agents, non-steroidal anti-inflammatory agents, prostanoids, antimicrobial agents, and anticancer agents.

Specific examples of steroidal anti-inflammatory agents include betamethasone phosphate, dexamethasone phosphate, prednisolone phosphate, hydrocortisone phosphate, prednisolone succinate, and hydrocortisone succinate.

Examples of non-steroidal anti-inflammatory agents include loxoprofen sodium, and diclofenac sodium.

Examples of prostanoids include Prostaglandin E₁ (PGE₁), while examples of antimicrobial agents include vancomycin, chloramphenicol succinate, latamoxef, cefpirome, clindamycin phosphate, and carumonam. Examples of anticancer agents include, but are not limited to, vincristin, and vinblastine.

In one exemplary process of the present invention, the intravenous nanoparticles are produced in the following manner: The low-molecular weight, water-soluble and non-peptide drug is first bound to the metal ion to make the agent hydrophobic. The drug is then dissolved or suspended, along with PLGA or PLA, in a water-miscible organic solvent. The resulting solution or suspension is added to an aqueous solution of a surfactant and the mixture is stirred to obtain the desired nanoparticles.

Examples of the water-miscible organic solvents for use in the present invention include, but are not limited to, acetone, acetonitrile, ethanol, methanol, propanol, dimethylformamide, dimethylsulfoxide, dioxane, and mixtures thereof.

Examples of the surfactants include polyoxyethylene polyoxypropylene glycols, polysorbates, polyoxyethylene octylphenyl ethers, lecithin, and polyvinylalcohol.

Preferably, the nanoparticles of the present invention so produced are purified by centrifugation, gel filtration, fiber dialysis, or ultrafiltration and are subsequently freeze-dried for storage to ensure the stability of PLGA or PLA as ingredient.

Upon freeze-drying, a stabilizing agent and an isotonizing agent are preferably added to the nanoparticles suspension so that the freeze-dried preparation can be resuspended for administration. Preferred examples of the stabilizing agent and isotonizing agent include sucrose and trehalose, which are preferably added in an amount (by weight) 5 times or greater than the amount of the nanoparticles.

The nanoparticles prepared in the above-described manner are intravenously administered to target various inflammatory sites, vascular lesions, infected sites, and malignant tumor tissues where the particles effectively accumulate and sustainedly release the encapsulated low-molecular weight, water-soluble and non-peptide drug over time to provide the desired biological activities for a prolonged period of time. This is where another advantageous feature of the nanoparticles of the present invention comes in: the metal ion acts to prevent the encapsulated low-molecular weight, water-soluble and non-peptide drug from bursting release out of the nanoparticles at an early stage after administration, thereby allowing the sustained release of the drug for a prolonged period of time.

Thus, in order for the nanoparticles to be usable as a medical formulation, it is important to control, depending on the intended purposes, the surface properties and the particle size of the nanoparticles, as well as the encapsulation rate and the release profile of the low-molecular weight, water-soluble and non-peptide drug. For instance, the surface properties of the nanoparticles can be controlled by using different types of surfactants.

Adjusting the particle size of the nanoparticles is important also because the distribution of the nanoparticles within living body is strongly influenced by the particle size. To this end, the size of the nanoparticles is adjusted by taking into account how well the particles accumulate to different lesion sites (e.g., inflammatory sites, vascular lesion sites, infected sites, and malignant tumor tissues). Specifically, the particle size can be adjusted by controlling the conditions during the preparation of the nanoparticles, including the rate at which the aqueous phase is stirred, the amount of the organic solvent used, and the rate at which the organic solvent is added to aqueous phase.

The efficiency of encapsulation of the low-molecular weight, water-soluble and non-peptide drug into the PLGA or PLA nanoparticles largely depends on the physical properties of the low-molecular weight drug. In general, hydrophilic (water-soluble) drugs tend to be incorporated into the PLGA or PLA nanoparticles less efficiently than hydrophobic drugs. For this reason, the low-molecular weight, water-soluble and non-peptide drug for use in the present invention needs to be bound to a metal ion to impart a hydrophobicity to the agent. Specifically, this is done by allowing the low-molecular weight, water-soluble and non-peptide drug to bind to a metal ion in such a manner that the drug forms water-insoluble precipitates.

For that purpose, such functional groups as phosphate and carboxyl, which are capable of binding to the metal ion, are preferably introduced into the molecules of the low-molecular weight, water-soluble and non-peptide drug. It is also required that any functional groups present in the drug molecules that do not participate in, or interrupt, the formation of the precipitation with the metal ion must be protected with proper protective groups.

Furthermore, the type and amount of the organic solvent used and the rate at which the organic solvent is poured also affect the particle size of the nanoparticles and therefore need to be optimized.

With respect to the PLGA and PLA that serve as ingredient of the nanoparticles, PLGA or PLA with different molecular weights may be used to adjust the rate at which the encapsulated low-molecular weight, water-soluble and non-peptide drug is released from the nanoparticles.

To evaluate the nanoparticles of the present invention, it is essential to construct in vitro or animal (in vivo) models suitable for the evaluation of PK/PD (pharmacokinetics/pharmacodynamics) of the nanoparticles.

As described above, the present invention has achieved a high encapsulation rate of the low-molecular weight, water-soluble and non-peptide drug into the PLGA or PLA nanoparticles by the use of metal ions to impart a hydrophobicity to the drug. The present invention allows the simple, industrial-scale production of the intravenous nanoparticles designed for the purpose of targeting drug delivery to target lesion sites where the particles can gradually release the drug over a prolonged period of time.

### EXAMPLES

The present invention will now be described in detail with reference to examples and test examples.

### Example 1

### Formation of Water-Insoluble Precipitates of Low-Molecular Weight, Water-Soluble and Non-Peptide Drug with Metal Ion

Compounds shown in Table 1 below were used to as the low-molecular weight, water-soluble and non-peptide drug having phosphate groups. Each compound was dissolved in a 0.2M Tris-HCl buffer solution (pH 7.8) to a concentration of 20 mM. The solution was then added to equal volume of 100 mM aqueous solutions of different metal ions. The turbidity of each of the resulting mixtures was observed.

The results are shown in Table 1 below.

TABLE 1
Formation of precipitates of low-molecular weight,
water-soluble and non-peptide drugs with metal ions

| | | Low-molecular weight, water-soluble and non-peptide drugs | | | | |
|---|---|---|---|---|---|---|
| | | | | | | Tris-HCl |
| | | | | | | buffer |
| | | Naphthyl- | betamethasone | Dexamethasone | riboflavin | solution |
| | | phosphate | phosphate | phosphate | phosphate | (0.1M/pH 7.8) |
| Metal | NiCl₂ | − | − | + | − | − |
| ions | CuCl₂ | − | +++ | +++ | +++ | − |
| | Zn(CH₃COO)₂ | +++ | +++ | +++ | +++ | − |
| | ZnCl₂ | +++ | +++ | +++ | +++ | − |
| | MgCl₂ | − | − | − | − | − |
| | FeCl₂ | +++ | +++ | +++ | +++ | − |
| | FeCl₃ | +++ | +++ | +++ | +++ | − |
| | 3N HCl | − | − | − | − | − |

The resulting mixture was evaluated as follows:
−: the compound was dissolved;
+: the mixture was slightly turbid;
++: the mixture was significantly turbid;
+++: the mixture was significantly turbid and a precipitation was formed.

As can be seen from the results of Table 1, a significant turbidity and precipitate formation were observed in each of the phosphate-containing compounds in the presence of zinc, iron (ferric or ferrous), or copper ion.

When the molar ratio of the betamethasone phosphate to zinc ion and the molar ratio of riboflavin phosphate to zinc ion were varied to examine the amounts of the resulting precipitates, the precipitate formation was most significant for each of the low-molecular weight compounds when the molar ratio with respect to zinc ion was approximately 1.

### Example 2

### Preparation of PLGA/PLA nanoparticles Encapsulating Steroids

Different steroids were dissolved in 100 μl water and the resulting solutions were each added to 500 μl of a 0.5M aqueous zinc acetate solution or 500 μl of a 0.5M aqueous ferrous chloride solution. Each mixture was centrifuged at 12,000 rpm for 5 min and the supernatant was discarded to obtain precipitates in the form of zinc-steroids or iron-steroids. To the precipitates, 500 μl of acetone, an acetone/acetonitrile mixture, or an acetone/ethanol mixture dissolved 20 mg PLGA or PLA (WAKO PURE CHEMICAL INDUSTRIES LTD.) were added respectively. To each of the resultant solutions, an aqueous solution of zinc acetate was added and the mixture was allowed to stand for 2 hours at room temperature. Subsequently, the solution (or suspension) was added at the rate of 1 ml/min via a 27G syringe to a 0.5% aqueous solution of Pluronic F68 (a nonionic high-molecular weight surfactant) stirring at 400 rpm, to give nanoparticles. The resultant nanoparticles were stirred for 1 to 2 hours at room temperature, and a 0.5M aqueous solution of EDTA (pH 8) was added (0.4 by volume). The mixture was then centrifuged at 20,000 G for 20 min. Following the removal of the supernatant, the residue was resuspended in water and the suspension was again centrifuged to wash the nanoparticles. The resulting nanoparticles were added to a 2N aqueous solution of NaOH to decompose PLGA/PLA, and the steroid content in the nanoparticles was determined by HPLC. Similarly, the amount of water-insoluble steroid was determined for the nanoparticles prepared by different method without metal ions.

Furthermore, precipitates formed by mixing 5 mg betamethasone phosphate with zinc were dissolved in varying volume of acetone and then encapsulation efficiency of betamethasone phosphate incorporated in the nanoparticles was determined in the same manner as described above.

The results are shown in Tables 2 and 3 below.

TABLE 2
Encapsulation of steroids into PLGA nanoparticles

| | Steroids | | | | |
|---|---|---|---|---|---|
| | | beta- | | | |
| | beta- | methasone | | | |
| | methasone | acetate | BDP | BP-Na | BP-Zn |
| Steroid/ | 0.01 | 0.15 | 0.47 | 0 | 2.03 |
| nanoparticle | | | | | |
| (wt %) | | | | | |

| | Steroids | | | | |
|---|---|---|---|---|---|
| | BP-Fe | DP-Na | DP-Zn | HP-Na | HP-Zn |
| Steroid/ | 1.15 | 0 | 1.15 | 0 | 1.05 |
| nanoparticle | | | | | |
| (wt %) | | | | | |

BDP: betamethasone dipropionate
BP: betamethasone phosphate
DP: dexamethasone phosphate
HP: hydrocortisone phosphate

TABLE 3
Effects of the volume of acetone on the encapsulation rate
of betamethasone phosphate into PLGA nanoparticles

| | Amounts of acetone(μl) | | | | | |
|---|---|---|---|---|---|---|
| | 500 | 700 | 900 | 1100 | 1300 | 1500 |
| Steroid/nanoparticle | * | 7.34 | 4.28 | 3.46 | 2.71 | 1.93 |
| (wt %) | | | | | | |

* Data not obtained because of particle aggregation

As shown in Table 2, the use of the precipitates of the steroid phosphates that were generated through the addition of zinc or ferrous ion (i.e., BP—Zn, BP—Fe, DP—Zn, and HP—Zn) significantly increased the encapsulation rate of the respective steroids into PLGA nanoparticles, as opposed to the cases of the steroid phosphates provided in the form of sodium salts, each of which showed substantially no incorporation into the nanoparticles.

Table 3 shows the encapsulation rates of betamethasone phosphate into PLGA nanoparticles obtained by varying the amount of the solvent, acetone, while maintaining the amounts of PLGA and betamethasone phosphate. As can be seen from these results, the nanoparticles formed aggregates in 500 μl or less of acetone. The particles on the other hand remained stably dispersed in 700 μl acetone while showing a high encapsulation rate of betamethasone phosphate into the nanoparticles. Although the nanoparticles were stably dispersed in 700 μl or more acetone, the encapsulation rates gradually decreased as the amount of acetone was increased.

### Example 3

### Steroid Release Profile from PLGA/PLA Nanoparticles

5 mg betamethasone phosphate was dissolved in 100 μl water and the solution was added to 500 μl of a 0.5M aqueous solution of zinc acetate. The mixture was then centrifuged at 12,000 rpm for 5 min and the supernatant was discarded to obtain a zinc-steroid precipitate. To the precipitate, 500 μl of acetone dissolved 20 mg of PLGAs or PLAs with different molecular weights was added. The solution was allowed to stand for 2 hours at room temperature and was subsequently added, at a rate of 1 ml/min with a 27 G syringe, to a 0.5% suspension of either Pluronic F68 (a nonionic high-molecular weight surfactant) or lecithin that had been stirred at 400 rpm. The resulting nanoparticles were stirred for 1 to 2 hours at room temperature. Following the addition of EDTA, the nanoparticles were subjected to ultrafiltration on Centriprep YM-10 (Amicon) for concentration and washing. The nanoparticles were then suspended in a mixture of FBS (fetal bovine serum)/PBS (v/v=1) at a 500 μg/mL PLGA concentration and, after a predetermined period of time, a 0.5M aqueous solution of EDTA (pH 8) was added (0.4 by volume). The suspension was then centrifuged at 20,000 G for 30 min and the supernatant was discarded. The residue was resuspended in water and the suspension was again centrifuged to wash the nanoparticles. The resulting nanoparticles were added to a 2N aqueous solution of NaOH to hydrolyze PLGA/PLA, and the steroid content in the nanoparticles was determined by HPLC.

As a control, nanoparticles encapsulating BDP (betamethasone dipropionate), a hydrophobic steroid, were prepared according to a method proposed by the present inventors in a previous patent application (Japanese Patent Application No. 2002-159190). The amount of the encapsulated steroid was determined in the same manner.

The results are shown in Table 4 below.

TABLE 4
Release of betamethasone from nanoparticles

| | Cumulative betamethasone released (%) | | | | | | |
|---|---|---|---|---|---|---|---|
| | 5 | Day | Day | Day | Day | Day | Day |
| PLGA/PLA | hrs | 1 | 2 | 4 | 8 | 11 | 20 |
| PLA*¹ | 27 | 53 | 64 | 79 | 97 | 98 | 100 |
| (Mw 14000) | | | | | | | |
| PLGA*² | 0 | 17 | 29 | 35 | 60 | 70 | 93 |
| (Mw 8000) | | | | | | | |
| PLGA*² | 0 | 11 | 18 | 34 | 47 | 53 | 62 |
| (Mw 13000) | | | | | | | |
| PLA*² | 0 | 12 | 13 | 25 | 28 | 30 | 38 |
| (Mw 9000) | | | | | | | |
| PLA*² | 0 | 3 | 4 | 8 | 10 | 14 | 31 |
| (Mw 14000) | | | | | | | |

*¹Nanoparticles prepared according to the method described in Japanese Patent Application No. 2002-159190
*²Nanoparticles prepared according to the method of the present invention

It was demonstrated that the nanoparticles encapsulating BDP (betamethasone dipropionate), a hydrophobic steroid, and prepared according to the method previously proposed by the present inventors (Japanese Patent Application No. 2002-159190) released a significant amounts of betamethasone at an early stage with approximately 90% or more of betamethasone having been released after 6 days. In contrast, the nanoparticles prepared according to the method of the present invention, in which the steroid's initial bursting release is significantly reduced, released the steroid in a more gradual manner and were able to release it over an extended period of time.

It has also been demonstrated that the nanoparticles made of PLGA or PLA with small molecular weights tend to release the steroid at an earlier stage and that the nanoparticles made of PLGA tend to release the steroid earlier than those made of PLA.

### Example 4

### Release Profile of Steroids from Nanoparticles Internalized by Macrophages

Macrophages were collected from the abdominal cavities of mice that had been stimulated by intraperitoneal administration of 1.5 ml of 10% proteose peptone. The cells were inoculated at 6×10⁵ cells/12 wells and were cultured overnight in Macrophage-SFM medium (Gibco). Subsequently, the culture medium was replaced, and the PLGA or PLA nanoparticles prepared according to the procedures described in Example 3 were added. The cells were incubated at 37° C. for another 2 hours. Subsequently, the cells were washed 8 times with PBS and the medium, and the amount of betamethasone in the medium was determined at pre-determined intervals by ELISA method.

As a control, nanoparticles encapsulating BDP (betamethasone dipropionate), a hydrophobic steroid, were prepared according to a method previously proposed by the present inventors (Japanese Patent Application No. 2002-159190) and were also added to the cells.

The results are shown in Table 5 below.

TABLE 5
Release profiles of betamethasone from
macrophages internalizing nanoparticles

| | Cumulative betamethasone released (%) | | | | | | | |
|---|---|---|---|---|---|---|---|---|
| | 2 | 4 | 10 | Day | Day | Day | Day | Day |
| | hrs | hrs | hrs | 1 | 2 | 3 | 5 | 7 |
| Control | 26 | 42 | 68 | 86 | 96 | 97 | 98 | 99 |
| nanoparticles*¹ | | | | | | | | |
| Nanoparticles of | 3 | 4 | 11 | 27 | 64 | 77 | 89 | 96 |
| the present | | | | | | | | |
| invention*² | | | | | | | | |

*¹Nanoparticles prepared using PLA (MW 14,000) (Japanese Patent Application No. 2002-159190)
*²Nanoparticles prepared using PLGA (MW 8,000)

It was demonstrated that the nanoparticles encapsulating BDP (betamethasone dipropionate), a hydrophobic steroid, and prepared according to the method previously proposed by the present inventors (Japanese Patent Application No. 2002-159190) had released most of betamethasone as early as after 2 days. In contrast, the nanoparticles prepared according to the method of the present invention showed a nearly linear release profile during the first 2 to 3-day period and continued to gradually release betamethasone for a succeeding period.

### Example 5

### Evaluation of Dispersion Stability of the Nanoparticles

The acetone solutions prepared according to the procedures described in Example 3 were added dropwise to aqueous solutions of different surfactants to obtain nanoparticles. The resulting nanoparticles were concentrated, washed, purified, and were then freeze-dried in sucrose solutions of varying concentrations. The freeze-dried nanoparticles were resuspended in water and particle sizes of the particles were measured using a light-scattering photometer.

All of the nanoparticles prepared by using aqueous solutions of different surfactants, namely, lecithin, polyoxyethylene polyoxypropylene glycols, and polysorbates, had substantially the same particle size. No significant differences were observed among the surfactants in the size and the dispersion stability of the nanoparticles, and in the encapsulation rate of betamethasone phosphate even when the concentrations of the surfactants were varied in the range from 0.01 to 1%.

In comparison, the nanoparticles prepared with a polyvinylalcohol solution were larger in size than those prepared with other surfactants and had a low encapsulation rate of betamethasone phosphate. It was also shown that the re-dispersibility of the freeze-dried nanoparticles by adding sucrose in an amount (by weight) more than 5 times the amount of the nanoparticles prior to freeze-drying the nanoparticles.

### Example 6

### Accumulation of Nanoparticles in Inflammatory Sites

Inflammation was induced by injecting 100 μl physiological saline containing 1% carrageenin in the left hind paw of male Lewis rats. After 4 hours, single dosages of rhodamine-encapsulating nanoparticles of two different sizes (200 nm and 500 nm) were injected into a tail vein. 2 hours after administration, the resultant leg edema was cut and cryostat sections were prepared. The tissue samples were observed with fluorescence microscopy.

As controls, one group was administered with physiological saline and another group with rhodamine alone.

The intensity of fluorescence observed in tissue sections was significantly higher in the group given the 200 nm nanoparticles than in the control group given physiological saline alone, indicating significant accumulation of the nanoparticles in the inflammatory sites.

No significant accumulation of the nanoparticles was observed in the group given rhodamine alone or the group administered with the 500 nm nanoparticles.

### Example 7

### Activity to Suppress Adjuvant-Induced Arthritis

Arthritis was induced in 7-week old Lewis rats, weighing 130 to 160 g and preconditioned for one week, by injecting, under ether anesthesia, 50 μl of incomplete Freund's adjuvant solution (DIFCO) containing 6 mg/mL M. Butyricum Desiccated (DIFCO) into the left hind paw. The animals were divided into groups so that there are no significant differences between the groups in terms of the volume of the left hind leg of the animals. 14 days after administration of M. Butyricum, a single dose of PLA nanoparticles encapsulating betamethasone phosphate was administered intravenously to one group.

As controls, single doses of betamethasone phosphate and phosphate-buffered saline (PBS) were subcutaneously administered to respective groups of rats and a single dose of limethason (MITSUBISHI PHARMA) was intravenously administered to another group.

The ability of the nanoparticles to suppress inflammation was analyzed by measuring the volume of the left hind legs before and 7 days after the administration of the drug using water displacement technique.

The results are shown in Table 6 below.

TABLE 6
Abilities of the nanoparticles to suppress
adjuvant-induced arthritis

| | Inflammation rate (%) after administration | | | | | | |
|---|---|---|---|---|---|---|---|
| | (# of days) *³ | | | | | | |
| Groups | 1 | 2 | 3 | 4 | 5 | 6 | 7 |
| Nanoparticles of the | 69 | 68.7 | 68.3 | 69 | 70.3 | 70.8 | 71.3 |
| present invention *¹ | | | | | | | |
| Limethason *² | 66.9 | 72 | 79.2 | 78.5 | 80 | 79 | — |
| Betamethasone | 68.3 | 76.5 | 79.2 | 81.7 | 88 | — | 84.8 |
| phosphate (300 μg) | | | | | | | |
| Betamethasone | 78.4 | 80 | 82.8 | 85.4 | 84.2 | 83 | 81.7 |
| phosphate (100 μg) | | | | | | | |
| Physiological saline | 100.8 | 98.1 | 98 | 96.7 | 96 | 95.5 | 96.2 |

*¹ Betamethasone phosphate-encapsulating nanoparticles were prepared using PLA (MW 14000). Nanoparticles were given in an amount corresponding to 100 μg Betamethasone phosphate.
*² Given in an amount corresponding to 100 μg dexamethasone phosphate.
*³ Inflammation rate was calculated by the following equation: Inflammation rate (%) = (measured leg volume − leg volume of normal rat un-injected adjuvant)/(leg volume before steroid administration − leg volume of normal rat un-injected adjuvant) × 100

As shown in Table 6, an anti-inflammatory effect comparable to that observed with the use of three times as much of the betamethasone phosphate was seen in the group administered with limethason, an anti-inflammatory agent already in clinical use, as early as 1 day after administration. As with the case where betamethasone phosphate alone was administered, the anti-inflammatory effect of limethason was gradually lost over time. In comparison, the PLA nanoparticles of the present invention encapsulating betamethasone phosphate exhibited, as early as 1 day after administration, a high anti-inflammatory effect comparable to that observed with limethason and continued to exhibit a strong effect over a succeeding 7-day period.

### Example 8

### Preparation of PLGA/PLA Nanoparticles Encapsulating PGE₁

1 mg of PGE₁ was dissolved in 20 μl ethanol and the solution was added to an 80 μl 0.5M aqueous solution of ferrous (or ferric) chloride. The mixture was then centrifuged at 12,000 rpm for 5 min and the supernatant was removed to obtain an iron-PGE₁ precipitate. To this precipitate, PLGA (WAKO PURE CHEMICAL INDUSTRIES, LTD.) or PLA (WAKO PURE CHEMICAL INDUSTRIES, LTD.) in acetone was added. An aqueous solution of zinc acetate was further added and the solution was allowed to stand for 2 hours at room temperature. Using a 27 G syringe, the solution (or suspension) was subsequently added, at a rate of 1 ml/min, to a 0.5% suspension of either Pluronic F68 (a nonionic high-molecular weight surfactant) or lecithin that had been pre-stirred at 400 rpm. The resulting nanoparticles were stirred for 1 to 2 hours at room temperature and a 0.5M aqueous solution of EDTA (pH 8) was added (0.4 by volume). The suspension was then centrifuged at 20,000 G for 20 min and the supernatant was discarded. The residue was resuspended in water and the suspension was again centrifuged to wash the nanoparticles. The resulting nanoparticles were dissolved in acetonitrile, followed by dilution with PBS. The amount of PGE₁ was then determined by ELISA method.

As described in Example 4, macrophages were allowed to uptake the PGE₁-encapsulating PLGA nanoparticles and the amount of PGE₁ contained in the medium was determined at intervals by ELISA.

The results are shown in Table 7 below.

TABLE 7
Release profile of PGE₁ from macrophages
internalizing nanoparticles

| | Cumulative PGE₁ released (%) | | | | | | | | |
|---|---|---|---|---|---|---|---|---|---|
| | 2 | 5 | 10 | Day | Day | Day | Day | Day | Day |
| | hrs | hrs | hrs | 1 | 2 | 3 | 4 | 6 | 8 |
| PGE₁-encap- | 22 | 42 | 60 | 75 | 90 | 95 | 98 | 99 | 100 |
| sulating | | | | | | | | | |
| nanopar- | | | | | | | | | |
| ticles*¹ | | | | | | | | | |

*¹Nanoparticles prepared using PLGA (MW 8,000)

The encapsulation rate of PGE₁ into the PLGA nanoparticles was approximately 0.1 to 1% by weight. As can also be seen from the results shown in Table 7, PGE₁ was continuously released from the nanoparticles for 8 days although the release profile was not as good as that for betamethasone phosphate, a steroidal anti-inflammatory agent.

### INDUSTRIAL APPLICABILITY

As set forth, the present invention provides intravenous PLGA or PLA nanoparticles that can encapsulate sufficient amounts of low-molecular weight, water-soluble and non-peptide drugs are less likely to burst at an early stage of administration, and are capable of releasing the drug for a prolonged period of time.

The intravenous nanoparticles of the present invention can be used to target various inflammatory sites, vascular lesion sites, infectious sites, and malignant tumor tissues and effectively accumulate in such sites or tissues where the encapsulated low-molecular weight, water-soluble and non-peptide drugs are released over time to exhibit their biological activities for a prolonged period of time. The potential medical impact that the nanoparticles of the present invention can bring about is thus significant.

1. Intravenous nanoparticles for targeting drug delivery and sustained drug release, characterized in that a low-molecular weight, water-soluble and non-peptide drug is made hydrophobic by metal ion and is encapsulated in nanoparticles formed with poly(lactic-co-glycolic acid) or poly(lactic acid), and a surfactant is applied to the surface of the nanoparticles of poly(lactic-co-glycolic acid) or poly(lactic acid).

2. The intravenous nanoparticles according to claim 1, wherein the particles have a diameter of 50 to 300 nm.

3. The intravenous nanoparticles according to claim 1, wherein the low-molecular weight, water-soluble and non-peptide drug has a molecular weight of 1000 or lower.

4. The intravenous nanoparticles according to claim 1, wherein the metal ion is any of zinc, iron, copper, nickel, beryllium, manganese, and cobalt.

5. The intravenous nanoparticles according to claim 1, wherein the low-molecular weight, water-soluble and non-peptide drug has a phosphate group to make the drug susceptible to hydrophobicization by the metal ion.

6. The intravenous nanoparticles according to claim 1, wherein the low-molecular weight, water-soluble and non-peptide drug has a carboxyl group to make the drug susceptible to hydrophobicization by the metal ion.

7. The intravenous nanoparticles according to claim 1, wherein the low-molecular weight, water-soluble and non-peptide drug is a steroidal anti-inflammatory drug, a non-steroidal anti-inflammatory drug, a prostanoid, an antimicrobial drug, or an anticancer drug.

8. The intravenous nanoparticles according to claim 1, wherein the surfactant is a polyoxyethylene polyoxypropylene glycol, a polysorbate, a polyoxyethylene octylphenyl ether, a lecithin, or a polyvinylalcohol.

9. A method for producing intravenous nanoparticles for targeting drug delivery and sustained drug release, comprising the steps of:
hydrophobicizing a low-molecular weight, water-soluble and non-peptide drug by the use of metal ion;
dissolving or suspending, along with a poly(lactic-co-glycolic acid) or a poly(lactic acid), the hydrophobicized drug in a water-miscible organic solvent; and
adding the resulting solution or the suspension to an aqueous solution of a surfactant to apply the surfactant to the surface of the nanoparticles.

10. The method for producing intravenous nanoparticles according to claim 9, wherein the particles have a diameter of 50 to 300 nm.

11. The method for producing intravenous nanoparticles according to claim 9, wherein the low-molecular weight, water-soluble and non-peptide drug has a molecular weight of 1000 or lower.

12. The method for producing intravenous nanoparticles according to claim 9, wherein the metal ion is any of zinc, iron, copper, nickel, beryllium, manganese, and cobalt.

13. The method for producing intravenous nanoparticles according to claim 9, wherein the low-molecular weight, water-soluble and non-peptide drug has a phosphate group to make the drug susceptible to hydrophobicization by the metal ion.

14. The method for producing intravenous nanoparticles according to claim 9, wherein the low-molecular weight, water-soluble and non-peptide drug has a carboxyl group to make the drug susceptible to hydrophobicization by the metal ion.

15. The method for producing intravenous nanoparticles according to claim 9, wherein the low-molecular weight, water-soluble and non-peptide drug is a steroidal anti-inflammatory drug, a non-steroidal anti-inflammatory drug, a prostanoid, an antimicrobial drug, or an anticancer drug.

16. The method for producing intravenous nanoparticles according to claim 9, wherein the surfactant is a polyoxyethylene polyoxypropylene glycol, a polysorbate, a polyoxyethylene octylphenyl ether, lecithin, or a polyvinylalcohol.

17. An anti-inflammatory/anti-rheumatoid drug containing nanoparticles encapsulating a water-soluble steroid according to claim 1, as an active ingredient.

18. The anti-inflammatory/anti-rheumatoid drug according to claim 17, wherein the water-soluble steroid is betamethasone phosphate.
